(19) United States
(12) Patent Application Publication
(10) Pub. No.: US 2026/0235503 A1
(43) Pub. Date: Aug. 13, 2026

(54) OPTICAL CONCENTRATION MEASURING DEVICE

(71) Applicant: Asahi Kasei Microdevices Corporation, Tokyo (JP)

(72) Inventors: Shota ISSHIKI, Tokyo (JP); Kyota SHIMAZAKI, Tokyo (JP)

(73) Assignee: Asahi Kasei Microdevices Corporation, Tokyo (JP)

(21) Appl. No.: 19/460,712

(22) Filed: Jan. 27, 2026

(30) Foreign Application Priority Data
Feb. 7, 2025 (JP) 2025-019613
Dec. 24, 2025 (JP) 2025-281137

(51) Int. Cl. G01N 21/3504; G01N 33/00

(52) U.S. Cl. CPC G01N 21/3504; G01N 2021/354; G01N 33/0027; G01N 2201/0636; G01N 2201/1211; G01N 2201/1214

(57) ABSTRACT

An optical concentration measuring device (1) including: a substrate (2); a light emitter (3) on a main surface (20) of the substrate; a light receiver (4); an environment measurement unit (7); and a housing defining the measurement space. The housing has light-guiding surfaces (52, 53, 54) that are reflective surfaces configured to guide at least a portion of the light emitted by the light emitter to the light receiver, and a support surface that is a reflective surface different from the light-guiding surfaces and has a surface roughness greater than a surface roughness of the light-guiding surfaces. The light-guiding surfaces include a first reflector and a second reflector. The first and second reflectors are on the main surface of the substrate opposite each other in an overhead view looking directly at the main surface of the substrate. The support surface is between the first reflector and the second reflector.

### CROSS-REFERENCE TO RELATED APPLICATION

The present application claims priority to and the benefit of Japanese Patent Application No. 2025-019613 filed Feb. 7, 2025, and Japanese Patent Application No. 2025-281137 filed Dec. 24, 2025, the entire contents of which are incorporated herein by reference.

### TECHNICAL FIELD

The present disclosure relates to optical concentration measuring devices.

### BACKGROUND

For example, optical concentration measuring devices that detect gas concentrations are used in various fields. An optical concentration measuring device includes, for example, a light source that emits infrared rays and a detector that detects infrared rays of a specific wavelength, in a single case. Gas to be detected is introduced into the case (for example, see Patent Literature (PTL) 1).

### CITATION LIST

### Patent Literature

PTL 1: JP 2004-138499 A

### SUMMARY

### Technical Problem

In order to realize an optical concentration measuring device having high measurement precision, temperature compensation is carried out when determining a measurement value. Typically, temperature compensation is carried out using correction data acquired during production. Further, as gas sensors become smaller, mirror coating is often applied to almost the entire interior of a housing, and light is often irradiated onto an environment device (environment measurement unit) mounted on a substrate. For example, when distortion of the housing occurs due to the external environment or the like, the amount of light irradiating the environment measurement unit, which carries out a measurement related to an environment of a measurement space where a measurement target is present, may fluctuate. As a result, factors such as an amount of temperature increase may change from those at the time of production, affecting temperature compensation and possibly decreasing the measurement precision of the optical concentration measuring device. For example, it is possible to decrease such effects by optimizing a position of the environment measurement unit on the substrate, but this limits placement locations and significantly decreases the degree of freedom in design.

In view of the above, it would be helpful to provide an optical concentration measuring device that allows a high degree of freedom in the placement of the environment measurement unit.

### Solution to Problem

(1) An optical concentration measuring device according to an embodiment of the present disclosure comprises:
a substrate;
a light emitter on a main surface of the substrate and configured to emit light;
a light receiver on the main surface and configured to receive at least a portion of the light emitted by the light emitter;
an environment measurement unit on the main surface and configured to carry out a measurement related to an environment of a measurement space where a measurement target is present; and
a housing defining the measurement space, wherein
the housing has light-guiding surfaces that are reflective surfaces configured to guide at least a portion of the light emitted by the light emitter to the light receiver, and a support surface that is a reflective surface different from the light-guiding surfaces and has a surface roughness greater than a surface roughness of the light-guiding surfaces,
the light-guiding surfaces comprise a first reflector and a second reflector,
the first reflector and the second reflector are on the main surface of the substrate opposite each other in an overhead view looking directly at the main surface of the substrate, and
the support surface is between the first reflector and the second reflector.
(2) As an embodiment of the present disclosure, (1), wherein
the environment measurement unit is a thermometer or a thermo-hygrometer.
(3) As an embodiment of the present disclosure, (1) or (2), wherein
a light guide including the light-guiding surfaces is at least a portion of inner surfaces of the housing provided so as to cover the main surface, and
the support surface is at least a part of a ceiling portion of the housing that is a flat surface facing the main surface.
(4) As an embodiment of the present disclosure, (3), wherein
the support surface is at least a part of a side surface portion that connects the ceiling portion of the housing and the main surface.
(5) As an embodiment of the present disclosure, (4), wherein
the support surface is 30% or more of the ceiling portion or 30% or more of the side surface portion.
(6) As an embodiment of the present disclosure, any one of (3) to (5), wherein
the light guide is a component integrated with the housing.
(7) As an embodiment of the present disclosure, any one of (1) to (6), wherein
a wavelength of the light is 2 μm to 10 μm.
(8) As an embodiment of the present disclosure, any one of (1) to (7), wherein
the light-guiding surfaces have an average surface roughness of 0.3 μm or less.
(9) As an embodiment of the present disclosure, any one of (1) to (8), wherein
the surface roughness of the support surface is 1 μm to 5 μm.
(10) As an embodiment of the present disclosure, any one of (1) to (9), wherein
the light emitter is configured to emit more than 20% of a total amount of the light at an emission angle greater than 45°.
(11) As an embodiment of the present disclosure, any one of (1) to (10), wherein
the light emitter is configured to emit less than 60% of a total amount of the light at an emission angle greater than 45°.

### Advantageous Effect

According to an embodiment of the present disclosure, an optical concentration measuring device having a high degree of freedom in placement of an environment measurement unit can be provided.

### BRIEF DESCRIPTION OF THE DRAWINGS

In the accompanying drawings:

FIG. 1 is a partially transparent perspective diagram of an optical concentration measuring device according to an embodiment of the present disclosure;

FIG. 2 is a diagram for explaining a light guide on an inner surface of a housing;

FIG. 3 is a diagram for explaining reflection at a support surface according to a present embodiment;

FIG. 4 is a diagram for explaining reflection at a support surface according to a comparative example; and

FIG. 5 is a diagram illustrating a configuration of a variation of an optical concentration measuring device.

### DETAILED DESCRIPTION

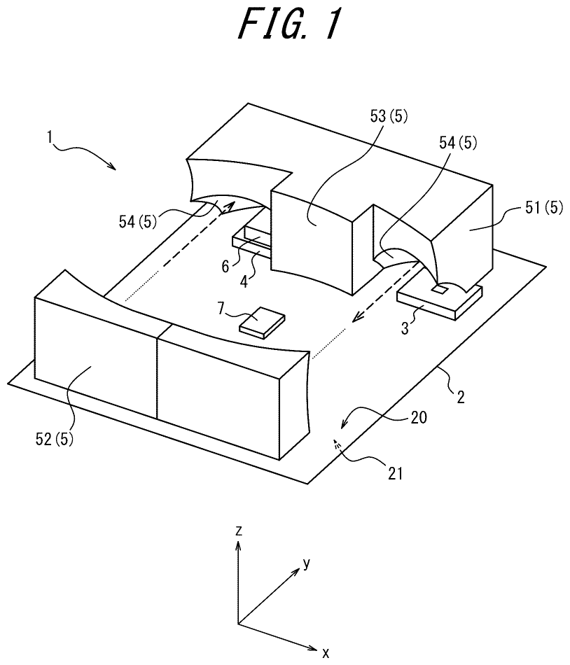

FIG. 1 is a partially transparent perspective diagram of an optical concentration measuring device 1 according to an embodiment of the present disclosure. The optical concentration measuring device 1 is a compact device, 20 mm×10 mm×10 mm as an example, and is also referred to as a gas sensor. According to the present embodiment, the optical concentration measuring device 1 is a non-dispersive infrared (NDIR) type device that measures the concentration of a gas to be detected based on infrared radiation transmitted through the gas that is introduced. The gas to be detected may be, for example, carbon dioxide, water vapor, methane, propane, formaldehyde, carbon monoxide, nitrogen monoxide, ammonia, sulfur dioxide, alcohol, or the like, or a mixture of any of these gases.

Here, the wavelength of the light (infrared light according to the present embodiment) may be 2 μm to 10 μm. The range of 2 μm to 10 μm contains many absorption bands specific to various gases, making it a wavelength range particularly suitable for use in gas sensors. For example, there is an absorption band for methane at a wavelength of 3.3 μm, for carbon dioxide at a wavelength of 4.3 μm, and for alcohol (ethanol) at a wavelength of 9.5 μm.

The configuration of the optical concentration measuring device 1 according to the present embodiment allows use as a light emitting and receiving device for applications other than gas detection. That is, disclosure content derived by replacing “optical concentration measuring device 1” as described above with “concentration measuring device”, “optical physical quantity measuring device”, “light receiving and emitting device”, “optical device”, or the like is included in the scope of the present disclosure. For example, the state of an optical path space can be detected (examples other than gas include the presence or absence or concentration of a specific component of a fluid). For example, the disclosure content can be used for a component detection device or a component concentration measuring device for a substance (for example, water or a body fluid) present in an optical path space between the light emitter and the light receiver. For example, when the substance present in the optical path space is blood, the component detection device or the component concentration measuring device can be used to measure glucose concentration in blood.

The component detection device or the component concentration measuring device can measure glucose concentration in blood by measuring absorption of light having a wavelength of 1 μm to 10 μm. In the measurement of glucose concentration in blood, measuring absorption of light in a 1.6 μm band, a 2.0 μm band, and a 10.0 μm band is preferred. A compact, high precision, and highly reliable non-invasive glucose concentration meter can be realized. Such a glucose concentration meter allows, for example, a diabetic patient to self-check blood sugar levels with good precision and without causing damage to the skin as would occur with an invasive method. Further, more accurate administration of medication (for example, insulin) can be achieved, based on the blood sugar levels checked.

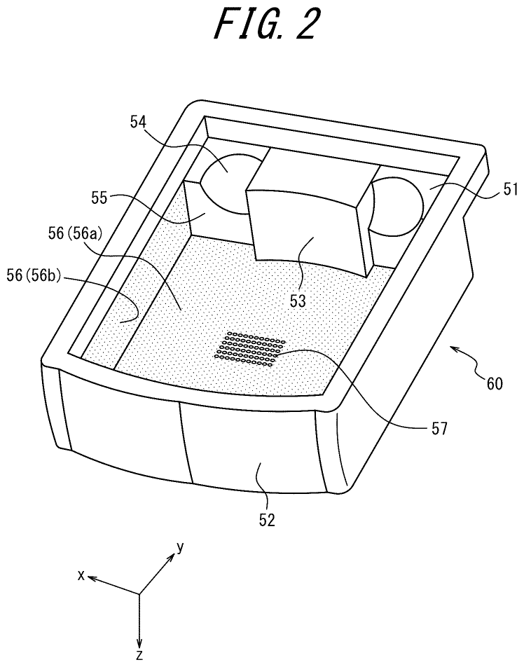

The optical concentration measuring device 1 includes a substrate 2, a light emitter 3, a light receiver 4, a light guide 5, and an environment measurement unit 7. As per the present embodiment, the optical concentration measuring device 1 may further include an optical filter 6. FIG. 1 illustrates an example configuration of the optical concentration measuring device 1 with a portion of the light guide 5 made transparent, and the light emitter 3, the light receiver 4, the optical filter 6, and the environment measurement unit 7 on a main surface 20 of the substrate 2 visible. According to the present embodiment, the main surface 20 is the surface with the largest area of the substrate 2 on which the light guide 5 is disposed. According to the present embodiment, the light guide 5 is at least a part of a housing 60 (see FIG. 2) provided to cover the main surface 20.

Hereinafter, as illustrated in FIG. 1, a Cartesian coordinate system is set so that the xy-plane is parallel to the main surface 20 of the substrate 2. The Cartesian coordinate system is used throughout FIG. 2 to FIG. 5. The z-axis direction is perpendicular to the main surface 20 of the substrate 2. The z-axis direction may also be referred to as the height direction. The x-axis direction and the y-axis direction are parallel to sides of the main surface 20 of the substrate 2. Hereinafter, the view of the front of the main surface 20 of the substrate 2 may also be referred to as the overhead view. The viewing direction in the case of the overhead view corresponds to the negative z-axis direction.

The substrate 2 is a board-like member that mounts components of the optical concentration measuring device 1 and makes electrical connections for mounted electronic components. According to the present embodiment, the substrate 2 is a printed circuit board (PCB). The material of the substrate 2 may be, for example, paper, glass cloth, ceramic, polyimide, liquid crystal polymer, or the like. The substrate 2 has the light emitter 3, the light receiver 4, and the light guide 5 mounted on the main surface 20. Further, the substrate 2 may have the optical filter 6 mounted on the main surface 20. Further, the substrate 2 may have the environment measurement unit 7 mounted on the main surface 20 as in the present embodiment. The substrate 2 may further mount other electronic components. For example, the substrate 2 may be provided with a controller that controls at least one of the light emitter 3 or the light receiver 4 on the main surface 20 or on a bottom surface 21. Further, the substrate 2 may be provided on the main surface 20 or the bottom surface 21 with an arithmetic unit that acquires an output signal from the light receiver 4 and a measurement value from the environment measurement unit 7 and executes a calculation to calculate gas concentration. The arithmetic unit may include at least one general-purpose processor that executes functions according to a program to be read and may include at least one dedicated processor specialized for a particular process. The dedicated processor may include an application specific integrated circuit (ASIC). The arithmetic unit may be integrated with the controller described above.

The light emitter 3 is a component that emits light used to detect the gas to be detected. The light emitter 3 is not particularly limited as long as the light emitter 3 outputs light including a wavelength that is absorbed by the gas to be detected. According to the present embodiment, the light emitted by the light emitter 3 is infrared light, but is not limited to this example. According to the present embodiment, the light emitter 3 is a light emitting diode (LED), and emits light from a light-emitting surface. The light-emitting surface is a surface of the light emitter 3 that is in contact with the gas and is made of an optically transparent material. According to the present embodiment, the light emitter 3 is a surface light source that emits light from a light-emitting surface, but is not limited to being a surface light source. As other examples, the light emitter 3 may be an organic light-emitting element, a lamp, a micro-electromechanical systems (MEMS) heater, or the like. The light emitter 3 is disposed on the main surface 20 of the substrate 2, opposite one of the secondary reflective surfaces 54, described below.

The light receiver 4 is a component that receives light transmitted through the gas that is introduced. The light receiver 4 receives at least a portion of the light emitted by the light emitter 3. The light receiver 4 is not particularly limited as long as the light receiver 4 is sensitive to a band of light that includes a wavelength absorbed by the gas to be detected. According to the present embodiment, light received by the light receiver 4 is infrared light, but is not limited to this example. According to the present embodiment, the light receiver 4 is a photodiode that receives light at a light-receiving surface. Here, the light-receiving surface is a surface of the light receiver 4 that is in contact with the gas and is made of an optically transparent material. As other examples, the light receiver 4 may be a phototransistor or thermopile, a pyroelectric sensor, a bolometer, or the like. The light receiver 4 converts received light into an electrical signal and outputs the converted electrical signal. The electrical signal is output to, for example, an arithmetic unit. The arithmetic unit that receives the electrical signal also acquires the measurement value from the environment measurement unit 7, and calculates the concentration of the gas to be detected based on light transmittance, the measured environment value, and the like. The light receiver 4 is disposed on the main surface 20 of the substrate 2, opposite the other one of the secondary reflective surfaces 54, described below.

According to the present embodiment, the light receiver 4 includes the optical filter 6 that has a wavelength selection function. The optical filter 6 transmits light emitted from the light emitter 3 and reflected by the light guide 5 to reach the light-receiving surface of the light receiver 4. Here, the optical filter 6 need only be provided on the optical path and need not be included in the light receiver 4. Further, the optical concentration measuring device 1 may be configured without the optical filter 6.

The light guide 5 is a member that guides at least a portion of the light emitted by the light emitter 3 to the light receiver 4. The light guide 5 is an optical system of the optical concentration measuring device 1. The light guide 5 includes optical members and configures the optical path from the light emitter 3 to the light receiver 4. In other words, the light guide 5 optically connects the light emitter 3 and the light receiver 4. Here, an optical member is, for example, a mirror. The optical members may also include a lens or the like.

According to the present embodiment, the light guide 5 includes a first reflector 51 and a second reflector 52. As illustrated in FIG. 1, the first reflector 51 and the second reflector 52 are connected to the main surface 20 of the substrate 2 and are opposite each other in the overhead view. Further, the first reflector 51 and the second reflector 52 may be held in a structure that has a function of holding the relative positions of the first reflector 51 and the second reflector 52, and the structure may be in the form of a connection to the main surface 20 of the substrate 2. According to the present embodiment, the housing 60 (see FIG. 2) is used as the structure that holds the relative positions of the first reflector 51 and the second reflector 52.

The first reflector 51 is configured to include a main reflective surface 53 that is a quadric surface, and two secondary reflective surfaces 54 that are each quadric surfaces. Here, quadric surfaces may include, for example, parabolic, ellipsoidal, spherical surfaces, and the like, and may have rotational axis symmetry. The second reflector 52 is configured to have two quadric surfaces. For example, the second reflector 52 may be configured to have two spherical surfaces joined together at a reflective surface opposite the first reflector 51. The first reflector 51 has three functions. The first function is to reflect light emitted from the light emitter 3 in the z-axis direction in the xy plane direction perpendicular to the z-axis direction. Here, the xy plane direction is a direction having a component in at least one of the x-axis direction and the y-axis direction. However, the xy plane direction may include a z-axis direction component. The second function is to cause multiple reflections of light with the second reflector 52. The third function is to reflect the multiple-reflected light to the light receiver 4.

One of the secondary reflective surfaces 54 near the light emitter 3 reflects at least a portion of the light emitted from the light emitter 3. The light reflected by the one of the secondary reflective surfaces 54 is reflected by the second reflector 52 and the main reflective surface 53 to traverse back and forth between the second reflector 52 and the main reflective surface 53 multiple times. The optical path is configured to traverse through a cell (internal space) between the light guide 5 and the substrate 2, where gas is introduced. The other one of the secondary reflective surfaces 54 near the light receiver 4 reflects light from the second reflector 52 and guides the light to the light receiver 4. Here, the light guide 5 may be configured to include a lens in a portion of the optical path. The one of the secondary reflective surfaces 54 near the light emitter 3 and the other one of the secondary reflective surfaces 54 near the light receiver 4 are both quadric surfaces, but need not be the same curved surface.

Material of the main reflective surface 53 and the secondary reflective surfaces 54 of the first reflector 51 and the reflective surface of the second reflector 52 may be, but is not limited to, metal, glass, ceramic, stainless steel, and the like. From the viewpoint of improving detection sensitivity, the material of these mirrors is preferably a material that has a low light absorption coefficient and high reflectance. Specifically, a resin housing coated with an alloy containing aluminum, gold, or silver, a dielectric, or a laminate thereof is preferred. From the viewpoint of reliability and aging, a resin housing coated with gold or an alloy layer containing gold is preferred. Vapor deposition or coating applied to a resin housing may provide improved productivity and lighter weight compared to forming with a metal material. Further, a difference in thermal expansion coefficient from the substrate 2 is reduced, thermal deformation is suppressed, and sensitivity fluctuation is suppressed.

FIG. 2 illustrates the housing 60 made of resin having an inner surface coated with the alloy layer described above. The housing 60 defines a measurement space. That is, the housing 60 is provided so as to cover the main surface 20 of the substrate 2, and constitutes a cell (measurement space, internal space) into which a gas is introduced. As illustrated in FIG. 2, the inner surface of the housing 60, which is the surface in contact with the internal space, is formed with the light guide 5 including the main reflective surface 53 and the secondary reflective surfaces 54 of the first reflector 51 and the second reflector 52. That is, according to the present embodiment, the light guide 5 is a component integrated with the housing 60. By integrating the light guide 5 with the housing 60, it is possible to omit a process of adjusting tolerances that would be required when incorporating a mirror of the light guide 5 as a separate component, thereby achieving high robustness.

Here, the light guide 5 has light-guiding surfaces that are reflective surfaces that guide light emitted by the light emitter 3 to the light receiver 4, and a support surface 56 that is a reflective surface different from the light-guiding surfaces. The support surface 56 is disposed between the first reflector 51 and the second reflector 52. The light-guiding surfaces include the first reflector and the second reflector. More specifically, the main reflective surface 53 and the secondary reflective surfaces 54 of the first reflector 51 and the reflective surfaces of the second reflector 52 are light-guiding surfaces. Further, as illustrated in FIG. 2, the support surface 56 includes a ceiling portion support surface 56a that is a flat surface facing the main surface 20 and that is at least a part of a ceiling portion of the housing 60. As illustrated in FIG. 2, the support surface 56 may include a side portion support surface 56b that is at least a part of a side surface portion that connects the ceiling portion of the housing 60 and the main surface 20. That is, the support surface 56 may be configured to include the ceiling portion support surface 56a, or may be configured to include both the ceiling portion support surface 56a and the side portion support surface 56b. As described in detail later, the surface roughness of the support surface 56 is greater than the surface roughness of the light-guiding surfaces (the main reflective surface 53 and the secondary reflective surfaces 54 of the first reflector 51 and the reflective surface of the second reflector 52).

Further, as illustrated in FIG. 2, the housing 60 may include a wall portion 55 that connects the secondary reflective surfaces 54 and the support surface 56. The wall portion 55 may be part of the support surface 56 or a light-guiding surface. Either the housing 60 or the light guide 5 may be selected depending on ease of working during production. Here, according to the present embodiment, the housing 60 includes the wall portion 55, but a configuration is possible without the wall portion 55, where the secondary reflective surfaces 54 and the ceiling portion of the housing 60 are smoothly connected.

Further, the housing 60 may be provided with holes 57 through which gas passes. Gas (air) is introduced into the internal space through the holes 57. In the example of FIG. 2, the holes 57 are provided in the ceiling portion of the housing 60, but the holes 57 may be provided in another location (for example, in the side surface portion).

Reference is now again made to FIG. 1. The environment measurement unit 7 carries out a measurement related to the use environment of the optical concentration measuring device. The environment measurement unit 7 is specifically a thermometer or a thermo-hygrometer. A measuring device (sensor) having a known configuration such as a thermometer or a thermo-hygrometer may be used. A temperature or a temperature and humidity measured by the environment measurement unit 7 may be output to the arithmetic unit, which may then execute temperature compensation for gas concentration calculation. A known calculation method may be used for temperature compensation.

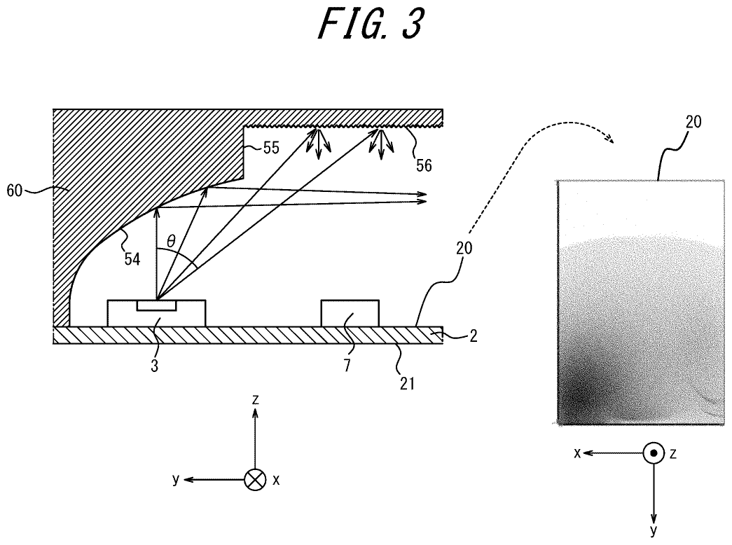

FIG. 3 is a diagram for explaining reflection at the support surface 56 according to the present embodiment. FIG. 3 illustrates a cross-section of one of the secondary reflective surfaces 54 and the support surface 56 taken so as to include light reflected by the secondary reflective surface 54 in the xy plane direction perpendicular to the z-axis direction. In the example of FIG. 3, light emitted from the light emitter 3 in the z-axis direction is reflected by the secondary reflective surface 54 in the xy plane direction perpendicular to the z-axis direction. Here, the xy plane direction is a direction having a component in at least one of the x-axis direction and the y-axis direction. However, the xy plane direction may include a z-axis direction component. Here, for example, the light emitter 3 emits more than 20% of a total amount of light at an emission angle greater than 45°. Further, the light emitter 3 emits less than 60% of the total amount of light at an emission angle greater than 45°. That is, the light emitter 3 emits 40% or more of the total amount of light at an emission angle of 0° or more and 45° or less. Further, the light emitter 3 emits 80% or less of the total amount of light at an emission angle of 0° or more and 45° or less. As an example, a light distribution of the light emitter 3 is a Lambertian distribution. In FIG. 3, the output angle is indicated by q, with light traveling straight in the height direction (z-axis direction) being the reference (0°). Of the light emitted by the light emitter 3, light having a large emission angle directly reaches the support surface 56 and is reflected, and then reaches the main surface 20 of the substrate 2. As described above, according to the present embodiment, the surface roughness of the support surface 56 is greater than the surface roughness of the light-guiding surfaces (the main reflective surface 53 and secondary reflective surfaces 54 of the first reflector 51 and the reflective surface of the second reflector 52). The reflection at the support surface 56 is dispersed by scattering, so that illuminance at the main surface 20 becomes uniform. The right diagram of FIG. 3 illustrates a distribution of illuminance on the main surface 20 as an overhead view. The portion of the diagram in the negative y-axis direction where light is not irradiated is a region of the main surface 20 beyond the reflective surface of the second reflector 52 in an overhead view of the main surface 20. The illuminance on the main surface 20 is uniform, and therefore the amount of light irradiated onto the environment measurement unit 7 is constant. An amount of light absorbed by mold resin that is a package component or a sensor itself of the environment measurement unit 7, or an amount of temperature increase caused by light absorption, are therefore constant regardless of the placement of the environment measurement unit 7. In other words, regardless of where the environment measurement unit 7 is placed on the substrate 2, distortion of the housing 60 after production does not change the amount of light absorbed or the amount of temperature increase in the environment measurement unit 7, or an amount of change is in a range that does not become a problem in actual use. Therefore, even when temperature compensation using correction data acquired during production continues to be used, measurement precision does not decrease.

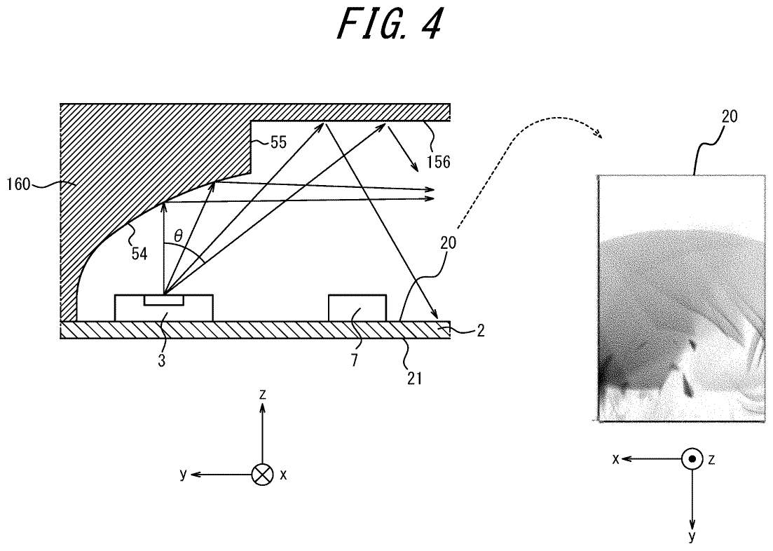

FIG. 4 is a diagram for explaining reflection at a support surface 156 according to a comparative example. In a housing 160 of the comparative example, the support surface 156 has the same surface roughness as the light-guiding surface. As illustrated in FIG. 4, light is reflected by the support surface 156 without being scattered and reaches the main surface 20, and therefore the illuminance on the main surface 20 becomes non-uniform and depends on the internal structure of the housing 160. The right diagram of FIG. 4 illustrates a distribution of illuminance on the main surface 20 in an overhead view, and it can be seen that the illuminance is biased. According to the comparative example, when distortion occurs in the housing 160, an amount of temperature increase and the like partially changes from the time of production, which affects a measured value of temperature and the like measured by the environment measurement unit 7. Such measured value fluctuation, such as temperature fluctuation, also affects a gas concentration calculation result after temperature compensation, and therefore the measurement precision of the optical concentration measuring device 1 decreases. Further, the amount of temperature increase and other changes vary depending on the placement location of the environment measurement unit 7 on the substrate 2.

As is clear from the comparison with the comparative example, the optical concentration measuring device 1 according to the present embodiment allows for a greater degree of freedom in the placement of the environment measurement unit 7. Here, it is preferable that the support surface 56 be 30% or more of the ceiling portion of the housing 60 or 30% or more of the side surface portion of the housing 60. Here, the ratio (30% or more) may be an area ratio. Even when the holes 57 are provided in the ceiling portion of housing 60 as illustrated in FIG. 2, it is preferable that the support surface 56 has an area of 30% or more of the entire area of the ceiling portion. The case where the support surface 56 is present on the side surface portion of the housing 60 is similar to the case where the support surface 56 is present on the ceiling.

Regarding the surface roughness, the light-guiding surfaces (the main reflective surface 53 and the secondary reflective surfaces 54 of the first reflector 51 and the reflective surface of the second reflector 52) have an average surface roughness of 0.3 μm or less. In contrast, the surface roughness of the support surface is preferably 1 μm to 5 μm. Here, the surface roughness is expressed as the root mean square height (Rq or Sq), which is the standard deviation from a reference surface.

The surface roughness is measured and confirmed using a measuring instrument or measuring method such as white light interference, a laser microscope, an atomic force microscope, or focus variation. The measurement region has an area corresponding to 50 μm square to 1000 μm square, and may be a rectangular, circular, or elliptical shape. When the area of one measurement region is smaller than 50 μm square to 1000 μm square, multiple regions may be measured so that the total area falls within this range. The measurement target is a surface on which a light-reflecting coating is applied, and when a protective layer is formed on the coating, the surface of the protective layer may be measured. Further, Rq may be calculated from any cross-section profile in the measurement region.

As described above, the optical concentration measuring device 1 according to the present embodiment can increase the degree of freedom in the placement of the environment measurement unit 7 due to the above configuration.

Although embodiments have been described based on the drawings and examples, it should be noted that a person skilled in the art may easily make variations and modifications based on the present disclosure. Therefore, it should be noted that such variations and modifications are included within the scope of the present disclosure.

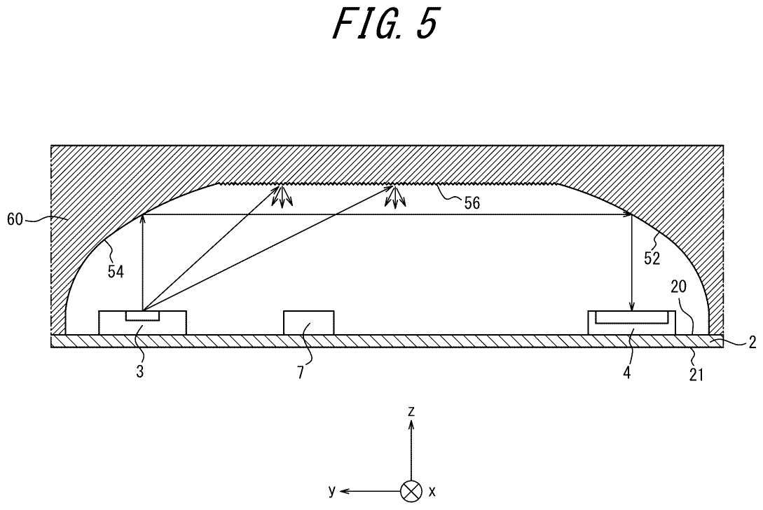

FIG. 5 is a diagram illustrating a configuration of a variation of the optical concentration measuring device 1. According to the variation, the light receiver 4 is disposed on the side of the second reflector 52, and the secondary reflective surface 54 near the light receiver 4 is also moved to the side of the second reflector 52. Even in the optical concentration measuring device 1 having such a configuration, the same effects can be obtained by using the same support surface 56 as in the embodiment described above.

1. An optical concentration measuring device comprising:
a substrate;
a light emitter on a main surface of the substrate and configured to emit light;
a light receiver on the main surface and configured to receive at least a portion of the light emitted by the light emitter;
an environment measurement unit on the main surface and configured to carry out a measurement related to an environment of a measurement space where a measurement target is present; and
a housing defining the measurement space, wherein
the housing has light-guiding surfaces that are reflective surfaces configured to guide at least a portion of the light emitted by the light emitter to the light receiver, and a support surface that is a reflective surface different from the light-guiding surfaces and has a surface roughness greater than a surface roughness of the light-guiding surfaces,
the light-guiding surfaces comprise a first reflector and a second reflector,
the first reflector and the second reflector are on the main surface of the substrate opposite each other in an overhead view looking directly at the main surface of the substrate, and
the support surface is between the first reflector and the second reflector.

2. The optical concentration measuring device according to claim 1, wherein the environment measurement unit is a thermometer or a thermo-hygrometer.

3. The optical concentration measuring device according to claim 1, wherein a light guide including the light-guiding surfaces is at least a portion of inner surfaces of the housing provided so as to cover the main surface, and
the support surface is at least a part of a ceiling portion of the housing that is a flat surface facing the main surface.

4. The optical concentration measuring device according to claim 3, wherein the support surface is at least a part of a side surface portion that connects the ceiling portion of the housing and the main surface.

5. The optical concentration measuring device according to claim 4, wherein the support surface is 30% or more of the ceiling portion or 30% or more of the side surface portion.

6. The optical concentration measuring device according to claim 3, wherein the light guide is a component integrated with the housing.

7. The optical concentration measuring device according to claim 1, wherein a wavelength of the light is 2 μm to 10 μm.

8. The optical concentration measuring device according to claim 1, wherein the light-guiding surfaces have an average surface roughness of 0.3 μm or less.

9. The optical concentration measuring device according to claim 1, wherein the surface roughness of the support surface is 1 μm to 5 μm.

10. The optical concentration measuring device according to claim 1, wherein the light emitter is configured to emit more than 20% of a total amount of the light at an emission angle greater than 45°.

11. The optical concentration measuring device according to claim 1, wherein the light emitter is configured to emit less than 60% of a total amount of the light at an emission angle greater than 45°.
